(19) United States
(12) Patent Application Publication
(10) Pub. No.: US 2008/0188701 A1
(43) Pub. Date: Aug. 7, 2008

(54) Process for producing light olefins

(75) Inventors: Guozhen Qi, Shanghai (CN); Zaiku Xie, Shanghai (CN); Siqing Zhong, Shanghai (CN); Xiaoqi Xin, Shanghai (CN); Huawen Wang, Shanghai (CN)

Correspondence Address: BIRCH STEWART KOLASCH & BIRCH, PO BOX 747, FALLS CHURCH, VA, 22040-0747, US

(73) Assignees: CHINA PETROLEUM & CHEMICAL CORPORATION, Beijing (CN); SHANGHAI RESEARCH INSTITUTE OF PETROCHEMICAL TECHNOLOGY SINOPEC, Shanghai (CN)

(21) Appl. No.: 12/068,343

(22) Filed: Feb. 5, 2008

(30) Foreign Application Priority Data
Feb. 7, 2007 (CN) 200710037235.8

(51) Int. Cl. C07C 1/00

(52) U.S. Cl. 585/641

(57) ABSTRACT

The present invention discloses a process for producing light olefins, comprising the steps of: i) contacting a feed comprising a monohalo-methane with a molecular sieve catalyst under the conditions: a reaction temperature in the range of from 350° C. to 600° C., a reaction pressure in the range of from 0.05 to 1.1 MPa (absolute), and a weight hourly space velocity of the monohalo-methane in the range of from 0.1 to 100 hour⁻¹, to give an effluent comprising ethylene, propylene, and hydrogen halide; and ii) isolating ethylene, propylene and hydrogen halide from the effluent.

### CROSS REFERENCE TO RELATED APPLICATIONS

The present application claims the benefit of the Chinese Patent Application No. 200710037235.8, filed on Feb. 7, 2007, which is incorporated herein by reference in its entirety and for all purposes.

### FIELD OF THE INVENTION

The present invention relates to a process for producing light olefins, in particular, to a process for producing light olefins through the conversion of monohalo-methane in the presence of a molecular sieve catalyst.

### BACKGROUND OF THE INVENTION

Light olefins, defined as ethylene and propylene herein, are important basic chemical feedstock, and the demand to them has been increasing. At present, ethylene and propylene are mainly produced through catalytic cracking or steam cracking of petroleum feeds. However, other processes for producing ethylene and propylene are paid more and more regard as the petroleum resource is being depleted and the prices of petroleum have been rising.

An important class of processes for producing light olefins from non-petroleum feeds involve the conversion of oxygenates such as lower alcohols (methanol, ethanol), ethers (dimethyl ether, ethyl methyl ether), esters (dimethyl carbonate, methyl formate) and the like to olefins, in particular, the conversion of lower alcohols to light olefins. Producing light olefins by methanol-to-olefins (MTO) process is a promising approach, since methanol can be produced at large scale from coal or natural gas via syngas. The literatures have disclosed a number of studies on the MTO process. See, for example, U.S. Pat. No. 6,166,282, CN1723262A, CN1166478A and CN1356299A.

However, such processes for producing olefins from coal or natural gas via syngas and methanol are relatively long in routing, and include syngas producing stage and methanol synthesizing stage, both of which require a large investment and a high operation cost, so that the economic performances of the processes depend largely on the prices of oil, coal or natural gas. Therefore, such processes suffer from poor economic performances.

Therefore, there still is a need for a novel economical process for producing light olefins from non-petroleum feedstock.

### SUMMARY OF THE INVENTION

The inventors made diligently studies, and as a result, have found that monohalo-methane may be converted efficiently into light olefins under selected conditions. On this basis, the invention has been completed.

An object of the invention is to provide a process for producing light olefins, comprising the steps of

i) contacting a feed comprising a monohalo-methane with a molecular sieve catalyst under the conditions: a reaction temperature in the range of from 350° C. to 600° C., a reaction pressure in the range of from 0.05 to 1.1 MPa (absolute), and a weight hourly space velocity of the monohalo-methane of from 0.1 to 100 hour⁻¹, to give an effluent comprising ethylene, propylene, and hydrogen halide; and

ii) isolating ethylene, propylene and hydrogen halide from the effluent.

The process for producing light olefins according to the invention has advantages of short routing, high olefin selectivity and good economic performances.

### DETAILED DESCRIPTION OF THE PREFERRED EMBODIMENTS

The process of the invention converts a monohalo-methane into light olefins. The monohalo-methane used as starting material in the process of the invention may be methyl chloride, methyl bromide or mixtures thereof, and is preferably methyl chloride from the viewpoint of cost.

Besides the monohalo-methane, the feed to the process of the invention comprises optionally one or more diluent(s). The addition of an amount of a diluent to the feed, on the one hand, will reduce the partial pressure of the monohalo-methane as starting material, thereby increasing selectivities to the light olefins, and on the other hand, might reduce the corrosion of a halogen-containing material to the plant during the reaction.

The diluent may be at least one selected from the group consisting of C₁-C₄ alkanes, such as methane, ethane, propane, n-butane, and iso-butane; C₁-C₄ alkanols, such as methanol, ethanol, n-propanol, iso-propanol, n-butanol, and iso-butanol; CO; CO₂; nitrogen; steam; and monocyclic aromatic hydrocarbons, such as benzene and toluene. Preferably, the diluent is at least one selected from the group consisting of C₁-C₄ alkanes, C₁-C₄ alkanols and steam, and more preferably steam. If the feed to the process of the invention comprises a diluent, then the volume ratio of the diluent to the monohalo-methane may be in the range of from 0.01:1 to 20:1, preferably from 0.1:1 to 10:1, and more preferably from 0.1:1 to 5:1.

In principle, the process of the invention may employ any of catalytic reactors known in the art, such as fluidized bed reactors, riser reactors, moving-bed reactors and fixed bed reactors. However, considering that the molecular sieve catalysts used in the process according to the invention have a characteristic of quick deactivation, it is preferable to employ a variety of dynamic bed reactors, for example, fluidized bed reactors, moving-bed reactors, riser reactors and the like. By using such a dynamic bed reactor, it is possible to achieve continuous regeneration and recycle of the catalysts. It is especially preferred for the process according to the invention to employ a fluidized bed reactor. The process according to the invention may be carried out in a single reactor or multiple reactors in parallel or in series.

The process according to the invention can be carried out under the following reaction conditions: a reaction temperature in the range of from 350 to 650° C., preferably from 350 to 600° C., more preferably from 400 to 600° C., still more preferably from 450 to 550° C., and most preferably from 450 to 500° C.; a weight hourly space velocity (WHSV) of the monohalo-methane in the range of from 0.1 to 100 hour⁻¹, preferably from 0.5 to 50 hour⁻¹, and more preferably from 1 to 20 hour⁻¹; a reaction pressure in the range of from 0.05 to 1.1 MPa (absolute), and preferably from 0.1 to 0.4 MPa (absolute).

The catalysts useful in the process according to the invention comprise one or more selected from aluminophosphate (ALPO) molecular sieves, silicoaluminophosphate (SAPO) molecular sieves, and metal substituted versions thereof. Preferably, the catalysts comprise a SAPO molecular sieve, such as SAPO-5, SAPO-11, SAPO-17, SAPO-18, SAPO-34, SAPO-35, SAPO-44, or SAPO-56, or a metal substituted version thereof, and more preferably, the catalysts comprise SAPO-34 or a metal substituted version thereof. The substituting metals include, but are not limited to, Ti, Zr, Cr, Mn, Fe, Co, Ni, Cu, Zn, Ga, Ge and Mg. The catalysts comprise optionally a matrix well known to those skilled in the art, such as silica, alumina, titania, zirconia, magnesia, thoria, silica-alumina, a variety of clay and the like, and mixtures thereof. The techniques for preparing suitable molecular sieve catalysts are well known by those skilled in the art.

The molecular sieve catalysts used in the process of the invention will be deactivated due to coking thereon. The deactivated catalysts can be regenerated by processes well known in the art, for example, by burning the coke in an oxygen-containing atmosphere. Prior to the regeneration, the deactivated catalysts removed from the reactor are optionally stripped to recover volatilizable carbonaceous materials adsorbed thereon.

In the process according to the invention, the feed comprising the monohalo-methane and optionally the diluent contacts with the molecular sieve catalyst in a reaction zone under selected reaction conditions, to form an effluent comprising ethylene and propylene. The feed may be fed into the reaction zone via, for example, a nozzle, a perforated distribution plate, or a pipe distributor. Before fed into the reaction zone, the feed may be in gas state and/or liquid state, and preferably in gas state. The feed may be fed into the reactor via a single feed inlet or multiple feed inlets. If multiple feed inlets are employed, the feeds fed into the reactor via the individual feed inlets may be the same or different in composition.

Ethylene, propylene and hydrogen halide as a by-product can be isolated from the effluent by processes known per se.

In an embodiment of the invention, the starting material is methyl chloride, and thus the main by-product generated in the process according to the invention will be HCl. In an aspect of this embodiment, the HCl by-product can be captured in a water-wishing quench tower in the separation unit, and then recycled to methyl chloride-preparing stage directly or after having been oxidized into Cl₂. The manner reusing the HCl by-product will depend on the process for preparing methyl chloride. The methods for isolating HCl and reusing it are known per se to those skilled in the art.

Without limited to any specific theory, it is believed that in the process of the invention, the monohalo-methane (CH₃—X) forms light olefin products through a mechanism similar to the mechanism through which methanol forms olefins, wherein the monohalo-methane forms at first intermediates, such as multi-methyl benzene, on the molecular sieve catalysts, and then the intermediates undergo continuously methylation and dealkylation to give light olefin products.

The process according to the invention has advantages of short routing, small investment, and high olefin selectivity. With the process of the invention, selectivity to light olefins may be as high as 84.35 wt %.

### EXAMPLES

The following examples are given for further illustrating the invention, but do not make limitation to the invention in any way.

### Examples 1 to 5

2 grams of SAPO-34 molecular sieve catalyst, which was prepared by spray drying process and comprised 50 wt % of SAPO-34 molecular sieve as active component and 50 wt % of alumina as matrix, and of which particles had particle sizes in the range of from 20 to 40 mesh, were mixed with 6 grams of quartz sand, of which particles had particle sizes in the same range. Then the mixed particles were loaded in a 316 stainless steel fixed bed reactor having a diameter of 14 mm so that the loaded particles were located in the thermostatic zone of the reactor. Methyl chloride as raw material was pre-heated to a temperature of about 150 to 200° C., and then mixed with steam as diluent in a volume ratio of methyl chloride to steam of 1:0.1, and then the mixture was fed into the reactor. The reactions were allowed to continue at a WHSV of methyl chloride of 1 hour⁻¹ under atmospheric pressure at reaction zone temperatures as shown in the Table I below. The reaction effluents were analyzed by a gas chromatographic instrument equipped with a thermal conductivity detector. The results obtained when the running time was 1 hour are shown in the Table 1.

TABLE 1

| | Example No. | | | | |
|---|---|---|---|---|---|
| | 1 | 2 | 3 | 4 | 5 |
| Reaction temperature, | 350 | 400 | 450 | 500 | 600 |
| ° C. | | | | | |
| Conversion of methyl | 28.46 | 42.34 | 75.53 | 83.24 | 89.8 |
| chloride, wt % | | | | | |

Selectivities of light olefins, calculated on carbon, wt %

| | | | | | |
|---|---|---|---|---|---|
| Ethylene | 28.31 | 35.7 | 55.68 | 57.36 | 60.15 |
| Propylene | 42.08 | 39.18 | 27.04 | 25.13 | 14.28 |
| C₄ olefins | 17.42 | 14.94 | 8.29 | 7.17 | 5.84 |
| Light olefins (ethylene + | 70.39 | 74.88 | 82.72 | 82.49 | 74.43 |
| propylene) | | | | | |

### Examples 6 to 8

Experiments were conducted according to the procedure of Example 3, except that reaction pressure and WHSV of the raw material were changed as set forth in the Table 2 below. The results obtained when the running time was 1 hour are shown in the Table 2.

TABLE 2

| | Example No. | | |
|---|---|---|---|
| | 6 | 7 | 8 |
| Reaction pressure (gauge), MPa | 0.1 | 0.3 | 1 |
| WHSV of methyl chloride, hour⁻¹ | 2 | 4 | 11 |
| Conversion of methyl chloride, wt % | 73.85 | 70.96 | 68.95 |

Selectivities of light olefins, calculated on carbon, wt %

| | | | |
|---|---|---|---|
| Ethylene | 50.95 | 48.79 | 47.59 |
| Propylene | 26.87 | 26.18 | 25.91 |
| C₄ olefins | 7.96 | 7.54 | 7.29 |
| Light olefins (ethylene + propylene) | 77.82 | 74.97 | 73.5 |

### Examples 9 to 12

Experiments were conducted according to the procedure of Example 3, except that the WHSVs of methyl chloride as shown in the Table 3 below were employed. The results obtained when the running time was 1 hour are shown in the Table 3.

TABLE 3

| | Example No. | | | |
|---|---|---|---|---|
| | 9 | 10 | 11 | 12 |
| WHSV of methyl chloride, hour⁻¹ | 0.1 | 20 | 50 | 100 |
| Conversion of methyl chloride, wt % | 78.71 | 74.82 | 70.43 | 67.96 |

Selectivities of light olefins, calculated on carbon, wt %

| | | | | |
|---|---|---|---|---|
| Ethylene | 52.87 | 54.35 | 55.02 | 56.7 |
| Propylene | 20.95 | 25.98 | 23.05 | 19.08 |
| C₄ olefins | 8.96 | 9.18 | 10.96 | 11.74 |
| Light olefins (ethylene + propylene) | 73.82 | 80.33 | 78.07 | 75.78 |

### Examples 13 to 20

Experiments were conducted according to the procedure of Example 3, except that the diluents and the volume ratios of diluent to methyl chloride as set forth in the Table 4 below were employed. The results obtained when the running time was 1 hour are shown in the Table 4.

TABLE 4

| | Example No. | | | | | | | |
|---|---|---|---|---|---|---|---|---|
| | 13 | 14 | 15 | 16 | 17 | 18 | 19 | 20 |
| Volume ratio of | 5:1 | 10:1 | 0.1:1 | | | | | |
| diluent to methyl | | | | | | | | |
| chloride | | | | | | | | |
| Diluent type | steam | steam | methane | propane | methanol | Isopropanol | nitrogen | benzene |
| Conversion of | 78.28 | 79.68 | 75.49 | 75.46 | 77.89 | 77.85 | 75.86 | 74.82 |
| methyl chloride, | | | | | | | | |
| wt % | | | | | | | | |

Selectivities of light olefins, calculated on carbon in converted methyl chloride, wt %

| | | | | | | | | |
|---|---|---|---|---|---|---|---|---|
| Ethylene | 56.7 | 56.85 | 54.86 | 54.83 | 56.18 | 56.04 | 54.05 | 50.49 |
| Propylene | 26.48 | 24.1 | 26.49 | 26.41 | 28.17 | 27.95 | 26.49 | 20.17 |
| C₄ olefins | 8.09 | 7.51 | 8.15 | 8.19 | 10.47 | 9.47 | 8.16 | 5.87 |
| Light olefins | 83.18 | 80.95 | 81.35 | 81.24 | 84.35 | 83.99 | 80.54 | 70.66 |
| (ethylene + propylene) | | | | | | | | |

### Example 21

An experiment was conducted according to the procedure of Example 3, except that methyl bromide was used as starting material. The results obtained when the running time was 1 hour are as follows: conversion of methyl bromide=64.57 wt %, selectivity to ethylene=60.50 wt %, selectivity to propylene=23.79 wt %, and selectivity to C₄ olefins=5.28 wt %.

### Example 22 to 25

Experiments were conducted according to the procedure of Example 3, except that the catalysts as set forth in the Table 5 below were employed. The results obtained when the running time was 1 hour are shown in the Table 5.

TABLE 5

| | Example No. | | | |
|---|---|---|---|---|
| | 22 | 23 | 24 | 25 |
| Catalyst | SAPO-11* | SAPO-18* | SAPO-56* | Ga-SAPO-34* |
| Conversion of | 62.8 | 75.4 | 72.89 | 76.25 |
| methyl chloride, | | | | |
| wt % | | | | |

Selectivities of light olefins, calculated on carbon, wt %

| | | | | |
|---|---|---|---|---|
| Ethylene | 5.96 | 54.15 | 24.18 | 50.36 |
| Propylene | 20.17 | 26.12 | 20.47 | 28.47 |
| C₄ olefins | 15.78 | 8.17 | 6.87 | 9.25 |
| Light olefins | 26.13 | 80.27 | 44.65 | 78.83 |
| (ethylene + | | | | |
| propylene) | | | | |

*The catalyst comprised 50 wt % of the indicated molecular sieve and 50 wt % of alumina and was prepared by spray drying process, and the particles of the catalyst had particle sizes in the range of from 20 to 40 mesh.

### Example 26

An experiment was conducted according to the procedure of Example 3, except that a mixture of methyl chloride and methyl bromide (1:1 by weight) was used as raw material. The results obtained when the running time was 1 hour are as follows: conversion of methyl chloride=71.34 wt %, conversion of methyl bromide=64.57 wt %, selectivity to ethylene=61.21 wt %, selectivity to propylene=18.02 wt %, and selectivity to C₄ olefins=5.76 wt %.

### Example 27

An experiment was conducted according to the procedure of Example 3, except that a fluidized bed reactor was used, and 75 grams of a catalyst, which was prepared by spray drying process, comprised 50 wt % of SAPO-34 molecular sieve and 50 wt % of alumina matrix, and had an average particle size of 78 microns, were charged into the reactor. The results obtained when the running time was 1 hour are as follows: conversion of methyl chloride=72.78 wt %, selectivity to ethylene=59.45 wt %, selectivity to propylene=28.14 wt %, and selectivity to C₄ olefins=7.99 wt %.

### Example 28

An experiment was conducted according to the procedure of Example 27, except that a riser reactor was used, reaction temperature was changed to 550° C., and WHSV of methyl chloride was changed to 72 hour⁻¹. The results obtained when the running time was 1 hour are as follows: conversion of methyl chloride=73.99 wt %, selectivity to ethylene=61.79 wt %, selectivity to propylene=19.23 wt %, and selectivity to C₄ olefins=9.09 wt %.

While the invention has been described with reference to exemplary embodiments, it will be understood by those skilled in the art that various changes and modifications may be made without departing from the spirit and scope of the invention. Therefore, the invention is not limited to the particular embodiments disclosed as the best mode contemplated for carrying out this invention, but the invention will include all embodiments falling within the scope of the appended claims.

What is claimed is:

1. A process for producing light olefins, comprising the steps of
i) contacting a feed comprising a monohalo-methane with a molecular sieve catalyst under the conditions: a reaction temperature in the range of from 350° C. to 600° C., a reaction pressure in the range of from 0.05 to 1.1 MPa (absolute), and a weight hourly space velocity of the monohalo-methane in the range of from 0.1 to 100 hour⁻¹, to give an effluent comprising ethylene, propylene, and hydrogen halide; and
ii) isolating ethylene, propylene and hydrogen halide from the effluent.

2. The process according to claim 1, wherein the monohalo-methane is methyl chloride, methyl bromide or a mixture thereof.

3. The process according to claim 1, wherein the contacting of the feed with the molecular sieve catalyst is carried out under the conditions: a temperature in the range of from 450° C. to 500° C., a weight hourly space velocity of the monohalo-methane in the range of from 1 to 20 hour⁻¹, a pressure in the range of from 0.1 to 0.4 MPa (absolute).

4. The process according to claim 1, wherein the feed further comprises a diluent.

5. The process according to claim 4, wherein the diluent is at least one selected from the group consisting of C₁-C₄ alkanes, C₁-C₄ alkanols, CO, CO₂, nitrogen, steam and monocyclic aromatic hydrocarbons, and the volume ratio of the diluent to the monohalo-methane is in the range of from 0.1:1 to 10:1.

6. The process according to claim 5, wherein the diluent is at least one selected from the group consisting of methane, ethane, propane, n-butane, iso-butane, methanol, ethanol, n-propanol, iso-propanol, n-butanol, iso-butanol and steam, and the volume ratio of the diluent to the monohalo-methane is in the range of from 0.1:1 to 5:1.

7. The process according to claim 1, wherein the molecular sieve catalyst comprises one or more selected from the group consisting of aluminophosphate molecular sieves, silicoaluminophosphate molecular sieves, and metal substituted versions thereof.

8. The process according to claim 7, wherein the molecular sieve catalyst comprises at least one selected from the group consisting of SAPO-5, SAPO-11, SAPO-17, SAPO-18, SAPO-34, SAPO-35, SAPO-44, SAPO-56, and metal substituted versions thereof.

9. The process according to claim 8, wherein the molecular sieve catalyst comprises SAPO-34 molecular sieve and/or a metal substituted version thereof.

10. The process according to claim 7, wherein the molecular sieve catalyst further comprises a matrix.

11. The process according to claim 1, which is performed in a fluidized bed reactor, a moving-bed reactor, a riser reactor or a fixed bed reactor.
